(19) United States
(12) Patent Application Publication
(10) Pub. No.: US 2010/0285046 A1
(43) Pub. Date: Nov. 11, 2010

(54) Nontoxic ricin mutant compositions and methods

(75) Inventors: Nilgun E. Tumer, Belle Mead, NJ (US); Xiao-Ping Li, Flemington, NJ (US); Marianne Baricevic, Lanoka Harbor, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Rutgers, The State University, New Brunswick, NJ (US)

(21) Appl. No.: 12/310,833

(22) PCT Filed: Sep. 5, 2007
(86) PCT No.: PCT/US2007/020113

§ 371 Date: Jul. 20, 2010

Related U.S. Application Data
(60) Provisional application No. 60/842,300, filed on Sep. 5, 2006.

(51) Int. Cl. A61K 39/00; A61K 31/7088; C07H 21/04; C07K 14/415; A61K 38/16; A61P 35/00

(52) U.S. Cl. 424/185.1; 514/44 R; 536/23.6; 530/377; 514/19.3

(57) ABSTRACT

Disclosed are nontoxic ricin mutants and uses in connection with vaccines and cancer therapy.

### CROSS-REFERENCE TO RELATED APPLICATIONS

This application claims the benefit of the filing date of U.S. Provisional Patent Application 60/842,300, filed Sep. 5, 2006, the contents of which are hereby incorporated by reference in their entirety.

### GOVERNMENTAL SUPPORT

Development of the invention was supported by a National Institutes of Health grant (AI59720). Therefore, the Government may have rights in the invention.

### BACKGROUND OF THE INVENTION

The plant toxins, ricin and abrin and the bacterial toxins, Shiga and Shiga-like toxins are type II ribosome inactivating proteins that inhibit protein synthesis by removing a highly conserved adenine from the a-sarcin/ricin loop (SRL) of the large rRNA (7, 8, 41). They consist of a catalytic A chain covalently joined by a disulfide bond to a cell binding B chain and are highly toxic to eukaryotic cells (13, 34, 41). Ricin naturally exists in the seeds of Ricinus communis (castor bean), a plant native to Asia, the Middle East and southern Europe (13, 34).

In the castor bean, ricin A and B chains are encoded by a single gene, which is translated into a preproprotein of 576 amino acids. The ricin precursor consists of a 35 residue N-terminal extension, which contains the signal sequence (13). The mature RTA, which consists of 267 residues, is joined to the 262 residue mature RTB by a 12 residue linker peptide (13). The signal peptide directs the protein into the endoplasmic reticulum (ER) where proricin is core glycosylated and disulfide bonds are formed within the protein (13). Four disulfide bonds form within the RTB sequence and the fifth one joins RTA with RTB in the ricin holotoxin.

The B-chain of ricin (RTB) is a lectin that binds galactose or N-acetylgalactosamine receptors on the surface of target cells and promotes subsequent endocytosis of the A-chain (RTA) (13, 34). After RTB binds to its receptor on the surface of animal cells, a portion of the endocytosed RTA reaches the Golgi complex. RTA is an N-glycosidase that depurinates ribosomes in the cytosol by removing a specific adenine (A4324 in rat 28S rRNA) from the highly conserved SRL in the large rRNA (7, 8). RTA undergoes retrograde transport from the Golgi to the endoplasmic reticulum (ER) and is thought to enter the cytosol from the ER (23).

The depurination of the SRL has been reported to interfere with the elongation factor 1 (eEF-1) dependent binding of amino acyl-tRNA to the ribosome, as well as the GTP-dependent binding of elongation factor 2 (eEF-2) and inhibit protein synthesis at the translocation step (27, 35). There is evidence that ricin induces apoptosis in a wide variety of animal cells by mechanisms other than protein synthesis inhibition (32). Ricin-induced apoptosis in HeLa cells was associated with oxidative stress, glutathione depletion and activation of the caspase 3 cascade, followed by downstream events leading to apoptotic cell death (32, 39).

Since ricin and many other AB-toxins are quite stable, one or a few molecules are sufficient to kill cells (13). RTA has been used in cancer therapy as the active moiety of immunotoxins selectively targeted to cancer cells (5). Due to its potent cytotoxicity and wide availability, ricin has been exploited as a biological weapon and an agent of bioterrorism (2, 19) and has been classified as a level B biothreat by the Centers for Disease Control and Prevention. Inhalation of small amounts of ricin aerosol can rapidly and irreversibly damage cells of the respiratory tract, leading to severe pulmonary incapacitation or death (3, 12).

### SUMMARY OF THE INVENTION

Some aspects of the present invention are directed to nontoxic ricin mutants. In some embodiments, the ricin mutant is altered relative to a mature ricin A chain toxin having an amino acid sequence of (RTA 1-267), and lacks the C-terminal 12-20 amino acid residues of RTA 1-267. In other embodiments, the ricin mutant is (RTA 1-267, G83D), (RTA 1-267, G140R), (RTA 1-267, A147P), (RTA 1-267, E208K), (RTA 1-267, P250L, A253V), (RTA 1-267, M255L, V256N), (RTA 1-267, I251A), (RTA 1-202, P202L) and (RTA 1-213, R213D).

A separate group of ricin mutants of the present invention, notwithstanding their nontoxicity, surprisingly and unexpectedly exhibit eucaryotic ribosome depurination activity at least about equal to that of the ricin A chain toxin having the amino acid sequence of RTA 1-267. In some embodiments, the mutant contains the mutation S215F or the double mutation P95L, E145K. In other embodiments, the mutant lacks RTA 253-267).

Nucleic acid molecules encoding the ricin mutants, and various constructs and cells, tissue or organisms containing same (e.g., transformed plants including edible plants) and methods of making the ricin mutants are also provided.

Other aspects of the present invention are directed to compositions containing the ricin mutant or the nucleic acid, and a carrier, and methods of using the mutants or nucleic acids, or compositions containing them. Cell damage, particularly endothelial cell (EC) damage, produced by toxins such as ricin A chain, is a danger for individuals who have contact with such toxins. Individuals that are in danger of such contact include members of the armed services, as well as civilians, who may be exposed to chemical weapons or terrorist devices. Thus, in some embodiments, the compositions are in the form of a vaccine, wherein the ricin mutant (or the nucleic acid) is present (or in the case of the nucleic acid, is expressed) in an amount effective to elicit an immune response to ricin A chain toxin in an animal such as a human. Thus, a related aspect of the present invention is directed to a method of eliciting an immune response to ricin A chain toxin, comprising contacting an animal with a ricin mutant (or nucleic acid encoding the ricin mutant of the present invention in an amount effective to elicit an immune response to ricin A chain toxin, wherein an immune response to ricin A chain toxin is elicited in the animal, e.g., human. Due to their nontoxic (e.g., disarmed or attentuated) properties, the mutants of the present invention may elicit an effective immune response.

In other embodiments, the compositions are formulated for use in treatment of cancer. Thus, a related aspect of the present invention is directed to a method of treating cancer, comprising administering to a cancer patient a therapeutically effective amount of a ricin mutant of the present invention. Due to their nontoxicity, the mutants of the present invention may be administered as a separate active entity. In other embodiments, they are conjugated to a ligand (such as a monoclonal antibody, or fragment or single chain binding fragment thereof) that specifically binds to cancer cells.

### BRIEF DESCRIPTION OF THE DRAWINGS

FIG. 1. Immunoblot analysis of RTA expression. Membrane fraction (15 μg) isolated from 10D600 of cells expressing preRTA or mutants containing a premature termination codon (A), a frameshift mutation (B), a single point mutation (C), or a double point mutation (D) was separated on a 12% SDS-polyacrylamide gel and probed with polyclonal anti-RTA (1:3000). The RTA standard (1.5 ng) was purified RTA (Sigma, St. Louis, Mo.). The blots were probed with the ER membrane marker Dpmlp as a loading control.

FIG. 2. Viability of cells expressing the preRTA and the mutant forms of RTA. Yeast cells were first grown in SD-Leu media supplemented with 2% glucose to OD600 of 0.3 and then transferred to SD-Leu supplemented with 2% galactose. At indicated hours post induction on SD-Leu media containing galactose (left), serial dilutions were spotted on SD-Leu plates supplemented with 2% glucose. The top two panels show the cell viability up to 12 h in cells expressing the wild type preRTA or harboring the empty vector.

FIG. 3. Ribosome depurination in yeast expressing preRTA and the mutant forms in vivo. Total RNA isolated after 6 h of growth on galactose was analyzed by dual primer extension analysis using two different end labeled primers, the depurination primer (Dep) used to measure the extent of depurination and the 25S rRNA primer (25S) used to measure the total amount of 25S rRNA (37). (A) Primer extension analysis of the mutants containing a premature termination codon, (B) a frameshift mutation or (C) a point mutation. Primer extension analysis of cells harboring the empty vector is shown as a control.

FIG. 4. Ribosome depurination by wild type RTA and mutants in vitro. (A) Total protein extracted from the cytosolic fraction of 10 ml of yeast cells expressing preRTA or the mutants was analyzed on a 12% SDS-polyacrylamide gel and probed with polyclonal anti-RTA (1:3000). The first lane is purified RTA standard (10 ng). (B) Ribosomes isolated from yeast cells were treated with either wild type RTA or the mutants, S215F, P95L-E145K and P250L-A253V extracted from the cytosolic fraction of yeast cells in vitro and the extent of depurination was determined by dual primer extension analysis (37). The first lane corresponds to the untreated ribosomes and the second lane corresponds to primer extension analysis with protein extracted from cells harboring the empty vector. (C) The extent of ribosome depurination was quantified using a PhosphorImager from three independent depurination experiments with the wild type and the mutant proteins extracted from yeast in vitro.

FIG. 5. Analysis of cell death and nuclear fragmentation in yeast expressing preRTA and the mutants. (A) Cells were stained with Evans blue or DAPI at 24 hours after induction and visualized using a Zeiss Axiovert 200 inverted microscope (40× magnification). The DAPI stained nuclei are shown enlarged 40 times relative to the yeast cells (B) the percentage of cell death at different hours (h) after induction was quantified and is represented as the mean+SD (n=3).

FIG. 6. Production of reactive oxygen species (ROS) in cells expressing preRTA or the mutants. (A) Yeast cells were sampled at 24 h post induction and stained using diaminobenzidine (DAB) at 24 h after induction. (B) The amount of H₂O₂ production was quantified using 2′,7′-Dichlorodihydrofluorescein diacetate (DCDHF-DA). The results are represented as the mean±SD (n=3).

### BEST MODE FOR CARRYING OUT INVENTION

The amino acid and corresponding nucleic acid sequence of the ricin A chain (RTA) are set forth below, and are designated SEQ ID NOS:1 and 2 respectively.

```
            -35 -30 -25 -20
          M K P G G N T I V I W M Y A V A T W L C
        ATGAAACCGGGAGGAAATACTATTGTAATATGGATGTATGCAGTGGCAACATGGCTTTGT
    1 ---------!---------!---------!---------!---------!---------!  60
      -15 -10 -5 1 5
          F G S T S G W S F T L E D N N I F P K Q
        TTTGGATCCACCTCAGGGTGGTCTTTCACATTAGAGGATAACAACATATTCCCCAAACAA
  61 ---------!---------!---------!---------!---------!---------! 120
                                  10 15 20 25
          Y P I I N F T T A G A T V Q S Y T N F I
        TACCCAATTATAAACTTTACCACAGCGGGTGCCACTGTGCAAAGCTACACAAACTTTATC
121 ---------!---------!---------!---------!---------!---------! 180
                                  30 35 40 45
          R A V R G R L T T G A D V R H E I P V L
        AGAGCTGTTCGCGGTCGTTTAACAACTGGAGCTGATGTGAGACATGAAATACCAGTGTTG
181 ---------!---------!---------!---------!---------!---------! 240
                                  50 55 60 65
          P N R V G L P I N Q R F I L V E L S N H
        CCAAACAGAGTTGGTTTGCCTATAAACCAACGGTTTATTTTAGTTGAACTCTCAAATCAT
241 ---------!---------!---------!---------!---------!---------! 300
                                  70 75 80 85
          A E L S V T L A L D V T N A Y V V G Y R
        GCAGAGCTTTCTGTTACATTAGCGCTGGATGTCACCAATGCATATGTGGTAGGCTACCGT
301 ---------!---------!---------!---------!---------!---------! 360
                                  90 95 100 105
          A G N S A Y F F H P D N Q E D A E A I T
        GCTGGAAATAGCGCATATTTCTTTCATCCTGACAATCAGGAAGATGCAGAAGCAATCACT
361 ---------!---------!---------!---------!---------!---------! 420
                                110 115 120 125
          H L F T D V Q N R Y T F A F G G N Y D R
        CATCTTTTCACTGATGTTCAAAATCGATATACATTCGCCTTTGGTGGTAATTATGATAGA
421 ---------!---------!---------!---------!---------!---------! 480
                                130 135 140 145
          L E Q L A G N L R E N I E L G N G P L E
        CTTGAACAACTTGCTGGTAATCTGAGAGAAAATATCGAGTTGGGAAATGGTCCACTAGAG
481 ---------!---------!---------!---------!---------!---------! 540
                                150 155 160 165
          E A I S A L Y Y Y S T G G T Q L P T T A
        GAGGCTATCTCAGCGCTTTATTATTACAGTACTGGTGGCACTCAGCTTCCAACTCTGGCT
541 ---------!---------!---------!---------!---------!---------! 600
                                170 175 180 185
          R S F I I C I Q M I S E A A R F Q Y I E
        CGTTCCTTTATAATTTGCATCCAAATGATTTCAGAAGCAGCAAGATTCCAATATATTGAG
601 ---------!---------!---------!---------!---------!---------! 660
                                  190 195 200 205
            G E M R T R I R Y N R R S A P D P S V I
        GGAGAAATGCGCACGAGAATTAGGTACAACCGGAGATCTGCACCAGATCCTAGCGTAATT
661 ---------!---------!---------!---------!---------!---------! 720
                                210 215 220 225
          T L E N S W G R L S T A I Q E S N Q G A
        ACACTTGAGAATAGTTGGGGGAGACTTTCCACTGCAATTCAAGAGTCTAACCAAGGAGCC
721 ---------!---------!---------!---------!---------!---------! 780
                                230 235 240 245
          F A S P I Q L Q R R N G S K F S V Y D V
        TTTGCTAGTCCAATTCAACTGCAAAGACGTAATGGTTCCAAATTCAGTGTGTACGATGTG
781 ---------!---------!---------!---------!---------!---------! 840
                                250 255 260 265
          S I L I P I I A L M V Y R C A P P P S S
        AGTATATTAATCCCTATCATAGCTCTCATGGTGTATAGATGCGCACCTCCACCATCGTCA
841 ---------!---------!---------!---------!---------!---------! 900
              267
          Q F *
        CAGTTTTAA
901 --------- 909
```

For purposes of this disclosure, the term “mature RTA” is used interchangeably with “RTA 1-267”, as numbered in SEQ ID NO:1. Mature RTA lacks the N-terminal 35-amino acid signal sequence shown in SEQ ID NO:1. The ricin mutants of the present invention are described by reference to RTA 1-267. The ricin mutants of the present invention are nontoxic in that they do not cause death of eucaryotic cells, such as yeast cells, as determined in accordance with the protocols described in the working examples. The ricin mutants of the present invention may also be referred to as attenuated or disarmed with respect to their native cytotoxicity. They are also altered in relation to RTA 1-267 at least in terms of having one of more amino acid substitutions and/or deletion of C-terminal amino acid residues. The ricin mutants of the present invention may or may not contain the N-terminal 35-amino acid signal sequence depicted in SEQ ID NO:1. In addition, they may be glycosylated or non-glycosylated.

In PNAS 103(7):2268-73 (2006), Vitetta, et al., explain that because the key amino acid residues involved in the ribotoxic site (Y80, Y123, E177, R180, N209 and W211) and the vascular leak syndrome-inducing site (L74, D75 and V76) have been identified, a reasonable strategy for developing a safe vaccine was to introduce a single mutation into each site to produce a totally nontoxic RTA molecule. The present Applicants have discovered that other sequences in RTA may be influential in the manifestation of toxicity of this toxin. The ricin mutants of the present invention, on the other hand, do not require mutation at any of these positions.

In some embodiments, the ricin mutants lack the C-terminal 12-20 amino acid residues of RTA 1-267. Examples include (RTA 1-248), which referring to SEQ ID NO:1, lacks amino acid residues 249-267. Other examples include, without limitation, (RTA 1-249), (RTA 1-250), (RTA 1-250, P250L), (RTA 1-251), (RTA 1-253), (RTA 1-254) and (RTA 1-255).

In other embodiments, the ricin mutants behave similarly to RTA 1-267 in that they exhibit eucaryotic ribosome depurination activity at least about equal to that of the ricin A chain toxin, as determined in accordance with the protocols described in the working examples. In this regard, they differ from as well as from known ricin mutants purported to be nontoxic. Such ricin mutants may contain one or more of the mutation S215F, the double mutation P95L, E145K, or deletion of the last 15 C-terminal amino acid residues (i.e., RTA 253-267). Specific examples of ricin mutants of the present invention that fall within this category include, without limitation, RTA 1-267, S215F; RTA 1-267, P95L, E145K) and RTA 1-252. The latter mutant RTA 1-267 clearly falls within the first described category of ricin mutants as well.

In yet other embodiments, the ricin mutants are altered relative to a ricin A chain toxin in that they contain the mutation G83D, G140R, A147P, E208K, 1251A, P202L or R213D, or the double mutation P250L, A253V or M255L, V256N. Specific examples of ricin mutants of the present invention that fall within this category include, without limitation, (RTA 1-267, G83D), (RTA 1-267, G140R), (RTA 1-267, A147P), (RTA 1-267, E208K), (RTA 1-267, 1251A), (RTA 1-202, P202L), (RTA 1-267, P250L, A253V), (RTA 1-267, M255L, V256N) and (RTA 1-213, R213D).

Other ricin mutants of the present invention may be obtained by introducing one or more additional mutations into the resulting amino acid sequence, provided that they are innocuous and do not alter the basic characteristics of the mutants including their nontoxicity, eucaryotic ribosome depurination activity, immunogenicity (in the case of use as a vaccine as described herein) or therapeutic activity (such as in the case of anti-cancer uses described herein). Such ricin mutants may be referred to as functional equivalents or analogs of the ricin mutants specifically disclosed herein, and may be obtained using standard techniques in the art.

In terms of functional equivalents, skilled artisans will appreciate that there is a limit to the number of changes that may be made within a defined portion of the ricin mutant containing one or more mutations specifically disclosed herein and still result in a mutant with acceptable levels of the desired activities. A functional equivalent ricin mutant is thus defined herein as those peptide(s) or polypeptide(s) in which certain, not most or all, of the amino acid(s) may be substituted. Since the peptides of the present invention are rather long, an intermediate number of additional changes to the remaining RTA sequence (in contrast to shorter peptides which have less tolerance for additional changes). For example, in PNAS103(7):2268-73 (2006), Vitetta, et al., state that perturbations of RTA sequences within the regions D124-G140 and L161-E185 could alter immunogenicity.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues, glutamate and aspartate are negatively charged molecules; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; may be functional equivalents for purposes of the present invention.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, polypeptide or peptide is generally understood in the art (Kyte and Doolittle, J. Mol. Biol. 157(1):105-32 (1982)). Certain amino acids may be substituted for other amino acids having a similar hydropathic index or score and still retain a similar biological activity. In making changes based upon the hydropathic index, amino acids whose hydropathic indices are within ±0.2, ±0.1 or within ±0.5 may be substituted.

The ricin mutants of the present invention may be prepared by alteration of the encoding wild-type RTA DNA (e.g., SEQ ID NO:2); taking into consideration also that the genetic code is degenerate and that two or more codons may code for the same amino acid. Nucleic acids encoding these immunogenic compositions also can be constructed and inserted into one or more expression vectors by standard methods (Sambrook, et al., In: Molecular Cloning: A Laboratory Manual, Vol. 1, Cold Spring Harbor Laboratory Press, Cold Spring Harbor, N.Y. 1989), for example, using PCR™ cloning methodology.

Yet other nontoxic ricin mutants that may be useful in the present invention, either as immunogens for vaccine compositions, or as an active component of a composition for cancer therapy, have mutations L127A (e.g., RTA 1-267, L127A) and D244A (e.g., RTA 1-267, D244A).

For compositions containing the ricin mutants to be useful as a vaccine, wherein the ricin mutants function as an immunogen, an immune response to the immunogen must be produced in at least one cell, tissue or animal (e.g., a human). Thus, the present invention provides vaccine or immunogenic compositions that contain the ricin mutant as the immunogen, a nucleic acid encoding the immunogen (e.g., an immunogen expression vector), or at least one cell expressing or presenting an immunogen. Vaccination via the compositions containing the ricin mutant encoding nucleic acid is generally achieved by transfecting or inoculating an animal with the nucleic acid, such that upon contact, animal target cells express the nucleic acid. Expression in vivo by the nucleic acid may be, for example, by a plasmid type vector, a viral vector, or a viral/plasmid construct vector.

In order to effect replication, expression or mutagenesis of a nucleic acid, the nucleic acid may be delivered (“transfected”) into at least one cell. The transfection of cells may be used, in certain embodiments, to recombinately produce one or more vaccine components for subsequent purification and preparation into a pharmaceutical vaccine. In other embodiments, the nucleic acid may be comprised as a genetic vaccine that is administered to an animal. In other embodiments, the nucleic acid is transfected into at least one cell and the cell administered to an animal as a cellular vaccine component.

The ricin mutant nucleic acids may be introduced into cells or organisms via a vector, which is generally regarded as a carrier nucleic acid molecule into which a nucleic acid sequence can be inserted for introduction into at least one cell where it can be replicated. The ricin mutant may be situated in the vector so as to be in operable association with one or more regulatory or other genetic elements, including promoters and enhancers that effectively directs the expression of the DNA segment in the organelle, cell type, tissue, organ, or organism chosen for expression, initiation signals and internal ribosome binding sites, multiple cloning sites, splicing sites, termination signals, polyadenylation signals, origins of replication, and selectable markers.

A variety of vectors may be useful in connection with the present invention, including plasmid vectors, viral vectors (e.g., adenoviral vectors, AAV vectors, AAV vectors, retroviral vectors (lentiviruses including HIV-1, HIV-2 and SIV), as well as viral vectors derived from vaccinia virus, sindbis virus, cytomegalovirus, and herpes simplex virus. The vectors may be targeted to specific target cells by means of a specific binding ligand.

Vectors containing the ricin mutant nucleic acids of the present invention may be delivered to organelles, cells, tissues or organisms to achieve administration of the genetic vaccine using known techniques. Representative techniques include direct delivery of DNA such as by injection, electroporation, calcium phosphate precipitation, DEAE-dextran followed by polyethylene glycol, direct sonic loading, liposome mediated transfection, receptor-mediated transfection, microprojectile bombardment, agitation with silicon carbide fibers, Agrobacterium-mediated transformation, PEG-mediated transformation of protoplasts and desiccation/inhibition-mediated DNA uptake. The targets may be stably or transiently transformed.

Representative host cells include prokaryotes and eucaryotes alike, including bacteria, viruses, yeast, plant cells and animal cells. As used herein, the terms “cell,” “cell line,” and “cell culture” may be used interchangeably. All of these terms also include their progeny, which is any and all subsequent generations, which may or may not be identical on account of deliberate or inadvertent mutations. A tissue may comprise a host cell or cells to be transformed with a nucleic acid encoding a vaccine component. The tissue may be part or separated from an organism, and may include adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, small intestine, spleen, stem cells, stomach, testes, anthers, ascite tissue, cobs, ears, flowers, husks, kernels, leaves, meristematic cells, pollen, root tips, roots, silk and, stalks.

In certain embodiments, the host cell or tissue may be comprised in at least one organism. In certain embodiments, the organism may be, but is not limited to, a prokayote (e.g., a eubacteria, an archaea) or a eucaryote, such as a plant, as would be understood by one of ordinary skill in the art. In some embodiments, a nucleic acid encoding a ricin mutant of the present invention is transformed and expressed in plants, particularly edible plants such as tomato, cucumber and banana. All or a part of the plant material may be used to prepare a vaccine, such as an oral vaccine. Such methods are described in U.S. Pat. Nos. 5,484,719; 5,612,487; 5,914,123; and 5,977,438.

The compositions may also contain at least one additional immunostimulatory agent or nucleic acids encoding such agents separately or as a fusion. Examples of such agents include additional immunogens, immunomodulators, antigen presenting cells and adjuvants. One or more of the additional agents may be covalently bonded to the antigen or an immunostimulatory agent. The immunogenic composition may be conjugated to or contain an HLA anchor motif amino acids.

The vaccine compositions of the present invention may vary in a few ways, including the active component (which may be present in neutral form or in the form of a pharmaceutically acceptable base or salt) and the manner in which it is packaged for contact with cells of the animal e.g., administration or delivery. For example, a nucleic acid encoding the ricin mutant may be formulated with an adjuvant that is a protein. The immunogen or nucleic acid may be encapsulated in a lipid or liposome. Even further, it may be in the form of a cellular vaccine wherein the cell has been transformed with the ricin mutant nucleic acid. Such cellular hosts may be in culture, tissue, organ or an organism. They are isolated and then contacted with the cells of the animal.

Immunomodulators may augment the immune response. A variety of immunomodulators are known in the art. They include cytokines (e.g., interleukin 1 (IL-1), IL-2, IL-3, IL-4, IL-5, IL-6, IL-7, IL-8, IL-9, IL-10, IL-11, IL-12, IL-13, IL-14, IL-15, IL-18, beta-interferon, alpha-interferon, gamma-interferon, angiostatin, thrombospondin, endostatin, GM-CSF, G-CSF, M-CSF, METH-1, METH-2, tumor necrosis factor, TGF-beta and LT); chemokines (e.g., RANTES, MCAF, MIP1-alpha, MIP1-beta and IP-10); immunogenic carrier proteins (e.g., hepatitis B surface antigen, keyhole limpet hemocyanin (KLH) and albumins e.g., bovine serum albumin (BSA), ovalbumin, mouse serum albumin and rabbit serum albumin); biological response modifiers (e.g., cimetidine (CIM; 1200 mg/d) (Smith/Kline, PA); low-dose cyclophosphamide (CYP; 300 mg/M²) (Johnson/Mead, N J), or a gene encoding a protein involved in one or more immune helper functions, such as B-7); and adjuvants (e.g., alum, used in about 0.05 to about 0.1% solution in phosphate buffered saline; synthetic polymers of sugars (Carbopol®)) used as an about 0.25% solution; aggregation of the immunogen e.g., with heat, pepsin-treated (Fab) antibodies to albumin, mixture with bacterial cell(s) such as C. parvum or an endotoxin or lipopolysaccharide components of Gram-negative bacteria, emulsion in physiologically acceptable oil vehicles, such as mannide mono-oleate (Aracel A) or emulsion with a 20% solution of a perfluorocarbon (Fluosol-DA®) used as a block substitute, muramyl dipeptide (N-acetylmuramyl-L-alanyl-D-isoglutamine [MDP]), polysaccharides and polyamine varieties of polysaccharides such as chitin and chitosan, BCG (bacillus Calmette-Guerin, an attenuated strain of Mycobacterium), BCG-cell wall skeleton (CWS), with or without trehalose dimycolate; amphipathic and surface active agents, e.g., saponin and derivatives such as QS21 (Cambridge Biotech); nonionic block copolymer surfactants; oligonucleotides; Quil A; lentinen; and detoxified endotoxins. In embodiments wherein the ricin mutant nucleic acid is contained in a cell, the adjuvant may be incorporated into or otherwise physically associated with or conjugated to the cell membrane.

The vaccine and cancer compositions of the present invention (or individual components thereof) may be purified to the degree desired or necessary, given the particular circumstances and/or the hosts to which it is administered. Purification of the ricin mutants of the present invention or the nucleic acids can be carried out in accordance with standard techniques. Protein purification techniques include precipitation with ammonium sulfate, PEG or antibodies, or by heat denaturation, followed by centrifugation; fractionation; chromatographic procedures e.g., partition chromatograph (e.g., paper chromatograph, thin-layer chromatograph (TLC), gas-liquid chromatography and gel chromatography), gas chromatography, high performance liquid chromatography, affinity chromatography, supercritical flow chromatography, ion exchange chromatography, gel filtration chromatography, reverse phase chromatography, hydroxylapatite chromatography, lectin affinity chromatography; isoelectric focusing and gel electrophoresis. The ricin mutant encoding nucleic acids may be purified on polyacrylamide gels or cesium chloride centrifugation gradients. Cells or other components of the vaccine may be purified by flow cytometry.

The compositions of the present invention contain a carrier, e.g., a pharmaceutically acceptable carrier. Representative examples of types of carriers that may be suitable for use in the present invention include solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes and buffers. The choice of carrier(s) depends on the mode of contact with the animal cells, e.g., solid, liquid or aerosol form, and whether it needs to be sterile for such routes of administration as injection. The manner of administration of the vaccines may be varied in accordance with acceptable medical practice. Suitable routes of administration may include intravenous, intradermal, intraarterial, intraperitoneal, intralesional, intracranial, intraarticular, intraprostatical, intrapleural, intratracheal, intranasal, intravitreal, intravaginal, intratumoral, intramuscular, subcutaneous, intravesicular, mucosal, intrapericardial, oral, rectal, nasal and topical administration.

Liquid carriers include water, ethanol, polyol (e.g., glycerol, propylene glycol and liquid polyethylene glycol) and lipids (e.g., triglycerides, vegetable oils, liposomes). Other ingredients may be included for the purposes of maintaining desired fluidity, particle size and isotonicity. (e.g., lecithin coatings, liquid polyols, lipids, surfactants such as hydroxypropylcellulose, sugars and sodium chloride). Solid (or partially solid) carriers include binders, excipients, disintegration agents, lubricants, flavoring agents, and combinations thereof. Examples include: as binders gum tragacanth, acacia, cornstarch and gelatin; as excipients dicalcium phosphate, mannitol, lactose, starch, magnesium stearate, sodium saccharine, cellulose and magnesium carbonate; as disintegrating agents corn starch, potato starch and alginic acid; as lubricants magnesium stearate; as sweetening agents sucrose, lactose and saccharin; and as flavoring agents peppermint, oil of wintergreen, cherry flavoring and orange flavoring.

Sterile injectable solutions (e.g., for parenteral administration) are prepared by incorporating the active components in the required amounts in an appropriate solvent as a carrier, optionally along with any other desired ingredient, followed by filter sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously filter-sterilized liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

In some embodiments, the composition may be formulated or oral administration, in which case it is typically in the form of solutions, suspensions, emulsions, tablets, pills, capsules (e.g., hard or soft shelled gelatin capsules), sustained release formulations, buccal compositions, troches, elixirs, suspensions, syrups or wafers.

In some embodiments, the composition may be formulated as an eye drop, nasal solution or spray, aerosol or inhalant. Nasal solutions are usually aqueous in nature, and isotonic or slightly buffered to maintain a pH of about 5.5 to about 6.5. Antimicrobial preservatives may be added.

Vaccination schedule and dosages may be varied on a patient by patient basis, taking into account, for example, factors such as the weight and age of the patient, the type of disease being treated, the severity of the disease condition, previous or concurrent therapeutic interventions, the manner of administration and the like, which can be readily determined by one of ordinary skill in the art. The quantity to be administered depends on the subject to be treated, including, e.g., the capacity of the individual's immune system to mount an immune response, and the degree of protection desired. The dosage of the vaccine will depend on the route of administration and will vary according to the size of the subject. Precise amounts of an active ingredient required to be administered depend on the judgment of the practitioner. Dosage amounts of the ricin mutants generally range from about 1 ug to about 250 ug or more, and in some embodiments from about 1 to 100 ug, or even about 10 to about 50 ug, or any range derivable therein.

Advantageously, the vaccines are administered to animals, e.g., humans prior to exposure or contact (confirmed or suspected) to ricin A chain toxin. However, administration may follow such exposure or contact.

Suitable regimes for initial administration and subsequent booster administrations may vary. Typically, an initial administration is followed by at least one booster. To achieve acceptable immunity, multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals, e.g., monthly. See, e.g., Vitetta, et al., PNAS 103(7):2268-73 (2006). Periodic boosters at intervals of 1-5 years, usually three years, will be desirable to maintain protective levels of the antibodies to the ricin mutants.

Cancers that may be amenable to treatment with the ricin mutants of the present invention include but are not limited to leukemias and lymphomas (e.g., B-lineage acute lymphoblastic leukemia, Hodgkin's lymphoma, non-Hodgkin's lymphoma, chronic lymphocytic leukemia, B-lineage lymphoma, blast crisis of chronic myelocytic leukemia, hairy cell leukemia, AIDS lymphoma, EBV-lymphoma, cutaneous T cell lymphoma), brain tumors, neuroblastoma, osteosarcoma, soft tissue sarcoma, breast cancer, prostate cancer, ovarian cancer, testicular cancer, melanoma, lung cancer (e.g., non small cell lung cancer), colon cancer, pancreatic cancer, head and neck cancer, gastrointestinal cancer, and leptomeningeal neoplasms. Although the mutants of the present invention are nontoxic, they are still effective in that they inhibit growth of cancer cells, particularly the mutants that exhibit depurinate eukaryotic ribosomes.

The ricin mutants may be administered alone or in the form of fusion proteins (e.g., recombinant protein or physical or chemical coupling) with a cancer cell-targeting ligand. Examples of suitable cell binding components include antibodies to cancer proteins. To further enhance internalization of the ricin mutant into the cytosol of the cancer cells, the ricin mutant may also be fused or coupled to the native ricin B chain, or a peptide of another multi-domain protein that possesses an internalization or translocation domain, e.g., Pseudomonas exotoxin, diphtheria, pokeweed antiviral protein (PAP), etc. Such peptide sequences are known in the art. See, e.g., U.S. Pat. No. 5,616,482 (diphtheria), U.S. Pat. No. 5,328,984 (Pseudomonas exotoxin) and U.S. Pat. No. 5,756,322 (PAP).

Since many cancer cells overproduce cytokine receptors, the targets for cancer therapy with the ricin mutants of the present invention may include growth factor receptors, differentiation antigens, or other less characterized cell surface antigens. Thus, effective targeting ligands include, but are not limited to, cytokines, cytokine subunits, antibodies or antibody subunits. Specifically, as used herein the term “targeting ligand” is defined to mean all monoclonal antibodies, monoclonal antibody fragments, single chain variable region polypeptides, and cytokines known for use in the production of immunotoxins and fusion toxins.

Antibodies having specificity for a cell surface protein may be prepared by conventional methods. The term “antibody” as used herein is intended to include fragments thereof which also specifically react with a cell surface component. Antibodies can be fragmented using conventional techniques and the fragments screened for utility in the same manner as described above. For example, F(ab′)₂ fragments can be generated by treating antibody with pepsin. The resulting F(ab′)₂ fragment can be treated to reduce disulfide bridges to produce Fab fragments. Chimeric antibody derivatives, i.e., antibody molecules that combine a non-human animal variable region and a human constant region may also be useful in the practice of the present invention, especially from the standpoint that they tend to be less immunogenic than non-chimeric antibodies. Chimeric antibody molecules can include, for example, the antigen binding domain from an antibody of a mouse, rat, or other species, with human constant regions. Techniques that are standard in the art may be used to make chimeric antibodies containing the immunoglobulin variable region which recognizes a cancer cell surface antigen. Monoclonal or chimeric antibodies specifically reactive against cell surface components can be further humanized by producing human constant region chimeras, in which parts of the variable regions, particularly the conserved framework regions of the antigen-binding domain, are of human origin and only the hyper-variable regions are of non-human origin. Such immunoglobulin molecules may be made by techniques known in the art.

Specific examples of targeting ligands include, but are not limited to, a monoclonal antibody, monoclonal antibody fragment, or single chain variable region polypeptide directed against the B43, CD2, CD3, CD4, CD5, CD7, CD13, CD14, CD19, CD22, CD24, CD25, CD30, CD33, CD40, CD45, CD72, TXU1, NXU1, TP-1, or TP-3 antigen. Specific cytokine ligands include, but are not limited to, GM-CSF, IL-2, IL-3, IL-4, IL-6, IL-7, IL-8, IL-9, IL-10, IL-12, EGF, FGF, PDGF and NGF.

For purposes of cancer treatment, the ricin mutants of the present invention may be formulated and administered in accordance with the teachings herein in connection with vaccines. Therapeutically effective amounts of the ricin mutants may vary according to factors such as the disease state, age, sex, and weight of the individual, and the ability of antibody to elicit a desired response in the individual. Generally, the dosage amounts effective for eliciting a therapeutic response range from about 0.1 to about 50 mg/m² or more, and in some embodiments from about 1 to about 30 mg/m², and in other embodiments from about 5 to about 20 or 25 mg/m² and subranges derivable therefrom. Likewise, the dosage regime may be adjusted to provide the optimum therapeutic response. For example, several divided doses may be administered daily or the dose may be proportionally reduced as indicated by the exigencies of the therapeutic situation. The dosage may be continuous (e.g., in the form of an infusion over 1-3 hours) or as a single bolus administration. Cancer treatment regimens using ricin A chain have been reported in Schnell, et al., Annals Oncol. 14:729-36 (2003); and Frankel, et al., Seminars in Cancer Biology 6:307-17 (1995) (and references cited in Schnell and Frankel).

Aspects of the present invention are now described in connection with the following non-limiting examples. Unless otherwise, specified, all parts are by weight.

The following examples describe a large-scale mutagenesis of preRTA in the yeast, Saccharomyces cerevisiae, and isolation and analysis of mutant forms of RTA based on their inability to kill yeast cells. The nontoxic RTA mutants were characterized with respect to their ability to depurinate ribosomes, inhibit translation and cause cell death. To gain insight into the mechanism of ricin induced cell death, hallmarks of apoptosis in cells expressing the wild type and the nontoxic forms of RTA, were examined. Apoptotic markers, such as chromatin condensation, nuclear fragmentation and ROS production were observed in yeast expressing the wild type RTA, but not in cells expressing the nontoxic mutants, even though they depurinated ribosomes and inhibited translation. These results, which are believed to be the first of their kind, provide evidence that ribosome depurination and translation inhibition alone are not sufficient for the cytotoxicity of ricin.

### Materials and Methods

Yeast expression vectors. The preRTA cDNA was constructed by synthesizing the signal sequence (38) (Genewiz, North Brunswick, N.J.) and ligating it to mature RTA in pRAIBI30 (30). The preRTA cDNA was then cloned into the yeast expression vector, YEp351, downstream of the galactose-inducible GAL1 promoter (45). The preRTA plasmid was transformed into Saccharomyces cerevisiae strain W303 [MATa ade2-1 trpl-1 ura3-1 leu2-3, 112 his3-11, 15 canl-100 (from B. Thomas, Columbia University, New York)] and transformants were selected on SD-Leu media containing 2% glucose.

Mutagenesis of preRTA. Plasmid DNA mutagenesis was carried out as previously described (16). Briefly, the preRTA plasmid was incubated with 7% hydroxylamine for 20 h at 37° C., and then precipitated and transformed into yeast. Yeast cells were plated onto SD-Leu supplemented with 2% glucose and replica plated onto SD-Leu, containing 2% galactose. The preRTA plasmid was isolated from the colonies, which were able to grow on galactose and retransformed into yeast to confirm that the resistance was due to the plasmid. Plasmids isolated from colonies expressing RTA were characterized by sequence analysis.

Analysis of preRTA expression. Yeast cells harboring the preRTA plasmid were grown on SD-Leu, containing 2% glucose to an A₆₀₀ of 0.3. Cells were pelleted at 2000 g for 5 min, resuspended in SD-Leu media containing 2% galactose and grown for 6 h to induce RTA expression. For immunoblot analysis, ER membrane fractions were isolated as previously described (36). The membrane fraction was dissolved in SDS buffer and heated at 37° C. for 10 minutes before loading onto a 12% SDS polyacrylamide gel. The blots were probed using polyclonal anti-RTA antibodies (1:3000) produced in rabbits (Covance Research Products, Denver, Pa.). The blots were then stripped for 30-45 min with 8M guanidine hydrochloride and reprobed with antibody to dolichol phosphate mannose synthase (Dpm1p; Invitrogen, Carlsbad, Calif.) (1:4000). Glycosylated and deglycosylated purified RTA standard was obtained from Sigma Aldrich (St. Louis, Mo.).

Analysis of growth rate. Yeast cells were grown in SD-Leu containing 2% glucose media to an A₆₀₀ of 0.3 and were then transferred to SD-Leu containing 2% galactose. Aliquots were taken every two h and the A₆₀₀ was recorded. Doubling times were calculated based on exponential growth between 4 and 10 h post induction.

Cell viability analysis. Yeast cells expressing preRTA or preRTA mutants were grown on SD-Leu, containing 2% glucose to an A₆₀₀ of 0.3 and then transferred to SD-Leu media containing 2% galactose to induce preRTA expression. A serial dilution of cells was plated on SD-Leu plates containing 2% glucose at 0, 4, 6, 10 and 12 h post-induction. Plates were incubated at 30° C. for approximately 48 h.

rRNA depurination assay. Dual primer extension analysis was conducted to quantify rRNA depurination as previously described (37). Briefly, 2 μg of total yeast RNA from cells expressing RTA was hybridized with 106 CPM of end labeled depurination primer [5′-AGCGGATGGTGCTTCGCGGCAATG-3′]. The second primer hybridized upstream of the depurination site close to the 5′ end of the 25S rRNA. To quantify the extent of depurination, the target RNA was initially hybridized in the presence of excess amounts (700 μmol) of the two [γ-³²P] ATP end-labeled negative strand primers. The depurination primer described above annealed 73-nt 3′ of the depurination site (A₃₁₃₇) on the 25S rRNA. The 25S control primer [5′-TTCACTCGCCGTTACTAAGG-3′] annealed 100-nt 3′ of the 25S rRNA 5′ end. To allow for accurate quantification, the labeled 25S control primer was diluted 1:4 with unlabeled 25S control primer. Superscript II-reverse transcriptase was used in the primer extension assay as above. Extension products for the control and depurination fragments (100-nt and 73-nt, respectively) were separated on a 7M urea 5% polyacrylamide denaturing gel, and visualized and quantified on a PhosphorImager (Molecular Dynamics, Sunnyvale, Calif.). The amount of total yeast RNA and rRNA used was previously determined to be in the linear range of detection.

Extraction of proteins from yeast and in vitro depurination assay. Yeast cells (50 ml) containing pre-RTA or nontoxic mutants were induced on galactose for 6 h. Cells were resuspended in 1× low salt buffer (20 mM HEPES-KOH, pH 7.6, 100 mM potassium acetate, 5 mM magnesium acetate, 1 mM EDTA, 2 mM dithiothreitol and 0.1 mM phenylmethylsulfonyl fluoride) and lysed using glass beads. Samples were centrifuged briefly to remove cell debris and glass beads. The supernatant was transferred to a new tube and centrifuged at 100,000 g for 30 minutes to remove cell membranes and ribosomes. The resulting supernatant (100 μl) was collected. Yeast ribosomes were isolated as previously described (44). Yeast ribosomes (15 μl) were incubated with RTA protein extracted from yeast (10 μl) in 10×RIP buffer (600 mM KCl, 100 mM Tris-HCl, pH 7.4 and 100 mM MgCl₂) at 30° C. for 30 minutes (44). 100 μl of 2× extraction buffer (240 mM NaCl, 50 mM Tris-HCl, pH 8.8, 20 mM EDTA and 2% SDS) was added and rRNA was extracted with phenol:chloroform and precipitated with ethanol. The rRNA was analyzed using the dual primer extension assay (37) as described above.

In vivo [³⁵S] methionine incorporation. Translation inhibition was measured by in vivo [³⁵S] methionine incorporation. Yeast cells were grown to an A₆₀₀ of 0.3 in SD-Leu-Met containing 2% glucose. Cells were then resuspended in SD-Leu-Met containing 2% galactose for 6 h to induce the expression of either wild-type preRTA or the mutant forms. At time zero, [³⁵S] methionine was added to induced cells. After minutes, 400 μl of yeast cells were removed for growth measurements and additional aliquots of 400 μl were assayed for methionine incorporation in duplicate as previously described (37). The CPM was normalized to the A₆₀₀ reading, and rates of translation were determined as CPM/A₆₀₀/minute. Final results were displayed as percentage of total translation in yeast harboring the empty vector.

Reactive Oxygen Species (ROS) production, cell death and nuclear fragmentation. Yeast cells were sampled at 0, 2, 4, 6, 10 and 24 h post-induction, stained with 0.05% Evans blue for 30 minutes and then destained with water for 10 minutes. Cells were counted using a Zeiss Axiovert 200 inverted microscope. The percentage of cell death was calculated by counting ˜800 total cells as described by Xu et al. (47). All experiments were assayed in triplicate.

To detect nuclear fragmentation, cells were resuspended in PBS buffer (20 mM sodium phosphate, 140 mM NaCl, pH 7.4) and stained with DAPI (4′,4-diaminido-2-phenylindole, 1 μg/ml) for 5 minutes at room temperature. After staining, cells were washed with water 5 times and observed under a Zeiss Axiovert 200 inverted microscope with the epi-fluorescence setting. The digital images were acquired with a Zeiss Axiocam digital camera and software for image archival and management (Axiovision 3.0, Carl Zeiss Vision GmbH). ROS staining was carried out with diaminobenzidine (DAB) (1 mg/ml) for 10 minutes, followed by washing with water 3 times (42). The stained cells were observed under a Zeiss Axiovert 200 inverted microscope as described above.

Intracellular production of H₂O₂ was detected using the oxidant sensitive probe 2′,7′-Dichlorodihydrofluorescein diacetate (DCDHF-DA) (Invitrogen, Carlsbad, Calif.) (4). Two μl of fresh 5 mM DCDHF-DA was added to 1 ml of yeast cell culture (107 cells) and incubated at 28° C. for 30 minutes. The cells were then washed twice in sterile distilled water and resuspended in 1 ml of 50 mM Tris-HCl pH 7.5. After adding 20 μl of chloroform and 10 μl of 0.1% SDS, the cells were incubated for 15 minutes and pelleted. The fluorescence of the supernatant was measured using a HTS700 Perkin Elmer Bioassay Reader (Wellesley, Mass.) with excitation at 490 nm and emission at 518 nm.

### Results

Random mutagenesis. The full length cDNA corresponding to preRTA, which consists of a 35 residue N-terminal extension and the 267 residue mature RTA was cloned into the yeast expression vector downstream of the GAL1 promoter, mutagenized using hydroxylamine and transformed into yeast. Cells were plated on media containing glucose and replica plated on galactose containing plates. Out of a total of 15,000 transformants screened, 128 (0.82%) were able to grow on galactose containing media. Immunoblot analysis showed that RTA expression was detected in 87 (68%) out of 128 colonies. Of the 87 colonies that showed detectable RTA expression, 37 expressed a protein of the same molecular weight as the wild type RTA and 50 expressed smaller forms of RTA. All 87 plasmids isolated were retransformed into yeast to confirm that the loss of cytotoxicity was due to the plasmid.

Referring to Table 1, Nucleotide sequence analysis identified a total of 35 different mutations that led to the loss of cytotoxicity.

TABLE 1
Characterization of nontoxic RTA mutants
obtained by random mutagenesis.

| | | | | Depurination | Translation | |
|---|---|---|---|---|---|---|
| | Protein | Number of | | (% of | (% vector | Doubling |
| Plasmid | Change | Occurrence | Cytotoxicity | wild-type) | control) | Time (h) |
| preRTA | | | Yes | 100 | 35 | 18 |
| Vector | | | No | 2.0 | 100 | 6.3 |
| control | | | | | | |
| Group I | | | | | | |
| NT1001 | Q19 stop | 1 | No | 5.0 | ND | ND |
| NT1002 | Q55 stop | 2 | No | 1.0 | ND | ND |
| NT1003 | Q112 stop | 2 | No | 1.0 | ND | ND |
| NT1004 | Q128 stop | 3 | No | 1.0 | ND | ND |
| NT1005 | G140 stop | 1 | No | 1.0 | ND | ND |
| NT1006 | S149 stop | 1 | No | 2.0 | ND | ND |
| NT1007 | Q160 stop | 3 | No | 12.3 | ND | ND |
| NT1008 | Q173 stop | 3 | No | 4.3 | ND | ND |
| NT1009 | S176 stop | 2 | No | 4.5 | ND | ND |
| NT1010 | Q182 stop | 2 | No | 3.4 | ND | ND |
| NT1011 | W211 stop | 4 | No | 3.7 | ND | ND |
| NT1012 | Q219 stop | 2 | No | 7.9 | ND | ND |
| NT1013 | Q223 stop | 3 | No | 4.3 | ND | ND |
| NT1014 | Q231 stop | 3 | No | 9.6 | 60 | 9.0 |
| NT1015 | Q233 stop | 6 | No | 6.7 | 59 | 8.7 |
| NT1016 | L248 stop | 2 | No | 15.8 | 58 | 7.0 |
| Group II | | | | | | |
| NT1021 | T77P + 4¹ | 1 | No | 0.4 | ND | ND |
| NT1022 | Y84T + 48 | 1 | No | 5.6 | ND | ND |
| NT1023 | F92S + 40 | 1 | No | 2.4 | ND | ND |
| NT1024 | R114D + 18 | 1 | No | 2.8 | ND | ND |
| NT1025 | P202L + 1 | 2 | No | 2.3 | ND | ND |
| NT1026 | R213D + 31 | 1 | No | 2.8 | ND | ND |
| NT1027 | R213D + | 1 | No | 3.2 | ND | ND |
| | 31² | | | | | |
| NT1028 | S215F + 6 | 1 | No | 5.5 | ND | ND |
| NT1029 | P250L + 1 | 1 | No | 5.1 | ND | ND |
| Group III | | | | | | |
| NT1031 | G83D | 6 | No | 41 | 62 | 12 |
| NT1032 | G140R | 2 | No | 5 | 93 | 9.1 |
| NT1033 | A147P | 3 | No | 33 | 69 | 10 |
| NT1034 | E177K | 3 | No | 5.6 | 73 | 9.8 |
| NT1035 | Δ I 184 | 1 | No | 8.2 | 69 | 9.0 |
| NT1036 | E208K | 2 | No | 29 | 58 | 10 |
| NT1037 | G212E | 9 | No | 19 | 88 | 6.9 |
| NT1038 | S215F | 2 | No | 110 | 32 | 15 |
| NT1039 | P95L- | 1 | No | 115 | 41 | 10 |
| | E145K | | | | | |
| NT1041 | P95L (by | | Yes | 149 | 34 | 26 |
| | PCR) | | | | | |
| NT1042 | E145K (by | | Yes | 108 | 27 | 18 |
| | PCR) | | | | | |
| NT1040 | P250L- | 1 | No | 5.2 | 100 | 7.7 |
| | A253V | | | | | |
| NT1043 | P250L (by | | Yes | 158 | 31 | 20 |
| | PCR) | | | | | |
| NT1044 | A253V (by | | Yes | 175 | 30 | 24 |
| | PCR) | | | | | |

The majority of the mutations were isolated multiple times from colonies present on different plates, indicating that the mutagenesis screen using hydroxylamine was saturated. The mutants were divided into three groups: Group I (NT1001-NT1016) contained 16 different mutations with a premature termination codon, resulting in a truncated form of the protein. Group II (NT1021-NT1029) contained 9 different frameshift mutations. In this group, the N-termini of the proteins were the same as preRTA, but the C-termini were different depending on the position of the frameshift mutation. The number of amino acids added to the C-termini before the stop codon are indicated in Table 1. Group III (NT1031-NT1044) consisted of 14 different point mutations that resulted in single amino acid changes in the protein. Only two mutants in this group (NT1039 and NT1040) contained double point mutations. To determine which mutation was necessary for the loss of cytotoxicity, single mutations were generated by site-directed mutagenesis. As shown in Table 1, expression of preRTA containing the single point mutations was toxic to yeast, indicating that both mutations are required simultaneously for the loss of cytotoxicity.

Additional data are shown in Table 2.

TABLE 2
Frequency of the mutations in preRTA

| | | Percentage |
|---|---|---|
| Base Pair Change | Number of Occurrence | (%) |
| C to T | 43 | 47 |
| C to A | 2 | 2 |
| C to G | 1 | 1 |
| G to A | 32 | 35 |
| G to C | 2 | 2 |
| T to A | 2 | 2 |
| Deletion of T | 3 | 3 |
| Deletion of A | 1 | 1 |
| Deletion of C | 2 | 2 |
| Deletion of G | 2 | 2 |
| Deletion of TAT | 1 | 1 |
| Addition of T | 1 | 1 |

Table 2 shows the frequency of the base pair changes, including the silent mutations. As expected for hydroxylamine mutagenesis, C to T or G to A transitions accounted for 80% of the total base pair changes. The frequency of other base pair changes was relatively low. The frequency of the deletions or additions was approximately 12%. Due to the high frequency of C to T changes, 11 out of 14 glutamines encoded by CAA/G in preRTA were changed to stop codons (TAA/G) resulting in premature termination.

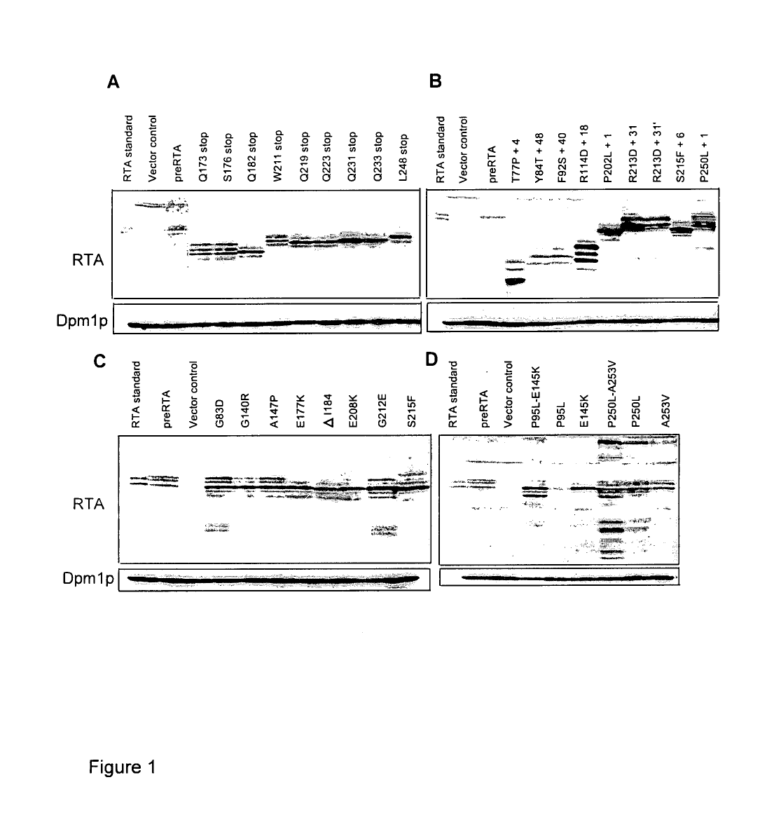

Wild type preRTA and the nontoxic mutants are expressed in yeast. Immunoblot analysis using polyclonal antibodies against RTA was used to examine protein expression in each mutant at 6 h post-induction. As shown in FIG. 1, purified RTA from Ricinus communis contains two bands. Based on comparison with the deglycosylated RTA standard, the upper band corresponds to the glycosylated from of RTA (data not shown). The endoplasmic reticulum (ER) membrane fraction isolated from yeast harboring the preRTA plasmid contained two bands that co-migrated with the purified RTA (FIG. 1), indicating that preRTA synthesized in yeast is processed the same way as RTA in plants. A very low level of protein was detected in the cytosolic fraction, indicating that the majority of RTA expressed in yeast is associated with the ER membranes (data not shown).

Immunoblot analysis indicated that all 39 mutants that contained premature termination codons (FIG. 1A), frameshift mutations (FIG. 1B) or point mutations (FIG. 10 and D) expressed detectable levels of RTA. The blot was reprobed with antibody against the ER membrane protein dolichol-phosphate mannose synthase (Dpm1p) as a loading control. The majority of the mutant proteins migrated on the SDS-polyacrylamide gels according to their predicted size. In several mutants, different forms of the protein were observed. The double mutant, P250L-A253V, contained both larger and smaller forms of the protein, suggesting possible aggregation and breakdown. In general, yeast cells carrying the nontoxic forms of RTA expressed higher levels of protein than cells carrying wild type or toxic forms (P95L) of preRTA. These results demonstrated that the loss of cytotoxicity was not due to the loss of protein expression.

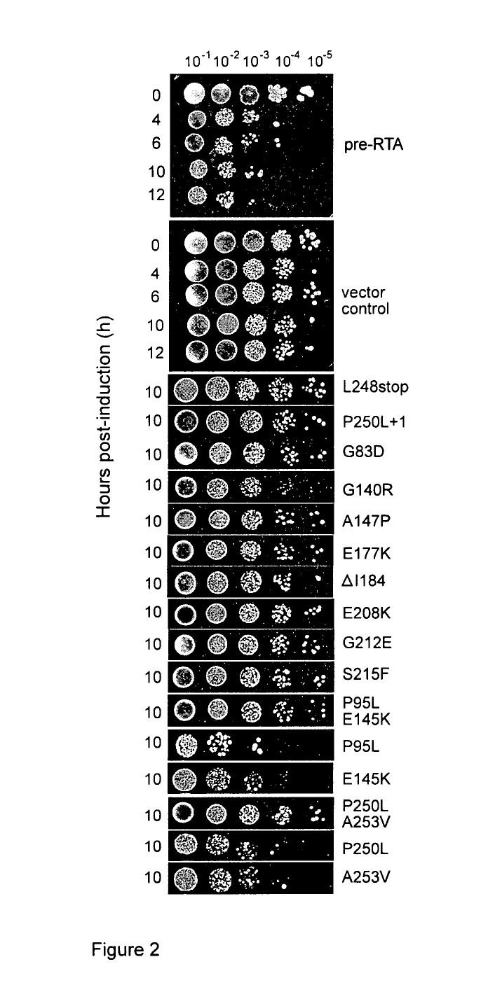

PreRTA mutants are not toxic to yeast cells. Irreversible growth inhibition was examined by conducting viability assays. Cells expressing preRTA or the nontoxic mutants were plated on glucose after induction in galactose for the indicated times (FIG. 2). Upon induction in yeast, the wild type RTA reduced the viability of cells by almost 3 logs at 10 h (FIG. 2, top panel). In contrast, the nontoxic RTA mutants exhibited minimal loss of viability at 10 h post induction. All nontoxic mutants analyzed exhibited similar viability as the cells harboring the empty vector. Only L248stop in group I and P250L+1 in group II, are shown because they had the shortest deletion at their C-termini. The two double mutants, P95L-E145K and P250L-A253V were nontoxic and did not reduce viability. However, the single mutants corresponding to each double mutant (P95L, E145K, P250L and A253V) reduced the viability of yeast cells (FIG. 2).

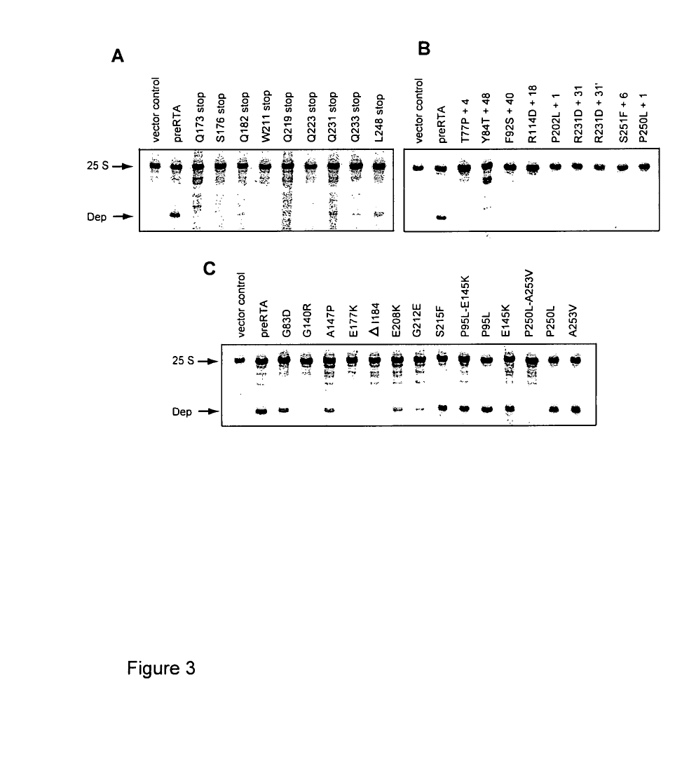

Nontoxic RTA mutants depurinate the rRNA. To determine if the reduced toxicity of the preRTA mutants was due to reduced depurination of ribosomes, total RNA was isolated from yeast cells expressing the wild type or the mutant forms of RTA and depurination of the rRNA was examined by a dual primer extension assay (37). As shown in FIG. 3A, ribosomes were depurinated in cells expressing preRTA. Cells expressing Q231stop, Q233stop, and L248stop showed a weak depurination band, indicating that these mutants retained a low level of ribosome depurination (FIG. 3A). The rest of the C-terminal deletion mutants did not depurinate ribosomes. None of the frameshift mutants showed any depurination (FIG. 3B). In contrast, 5 of the 10 point mutants depurinated yeast ribosomes in vivo (FIG. 3C). The depurination assay was repeated several times with all mutants and the extent of depurination calculated from independent experiments was averaged in Table 1. As shown in Table 1, the S215F and the double mutant, P95L-E145K, depurinated ribosomes at 110% and 115%, respectively. These results indicated that both mutants depurinated ribosomes at a similar level as the wild type preRTA in vivo, but unlike the wild type preRTA, they were nontoxic (Table 1) and did not reduce the viability of yeast cells (FIG. 2).

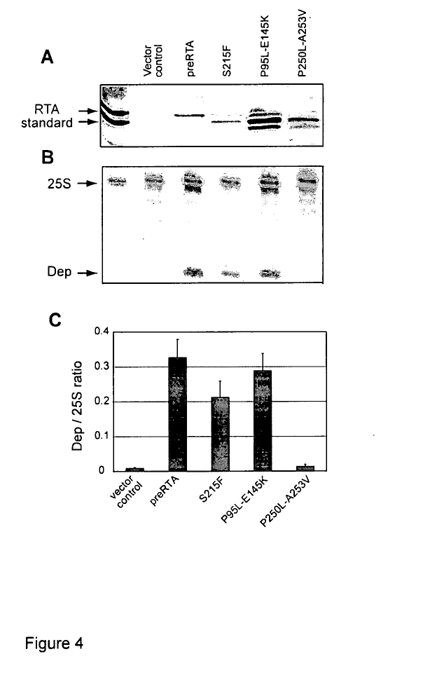

To determine if the mutant proteins were enzymatically active in vitro, the S215F and P95L-E145K mutants were extracted from the cytosolic fraction of yeast cells. These mutants were selected since they depurinated ribosomes at a similar level as the wild type RTA in vivo. Since P250L-A253V was nontoxic and did not depurinate ribosomes in vivo, it was used as a control for the in vitro depurination experiments. Purified yeast ribosomes were treated with similar amounts of the wild type and the mutant proteins isolated from yeast (FIG. 4A) and ribosome depurination was examined by dual primer extension analysis. As shown in FIG. 4B, the wild type RTA extracted from yeast depurinated yeast ribosomes in vitro. Both S215F and P95L-E145K depurinated yeast ribosomes in vitro, while P250L-A253V was not able to depurinate ribosomes. The extent of ribosome depurination quantified from three independent depurination experiments is shown in FIG. 4C. The in vitro depurination results were similar to those obtained in vivo and demonstrated that S215F and P95L-E145K were catalytically active, while P250L-A253V was not active.

Ribosome depurination results in translation inhibition. To determine if ribosome depurination correlated with translation inhibition, total translation in cells expressing preRTA compared with control cells harboring the empty vector was examined. Translation rates were determined by measuring the slope of the [³⁵S] methionine incorporation curve and expressed as percent of the translation rate in cells harboring the empty vector. As shown in Table 1, in cells expressing the wild type preRTA, the rate of translation was reduced to 35% of the rate of translation in cells harboring the empty vector (100%). Total translation was not inhibited in yeast expressing the RTA mutants that did not depurinate ribosomes. In contrast, total translation was inhibited in cells expressing S215F or the double mutant, P95L-E145K, which depurinated ribosomes (Table 1). These results demonstrated that translation inhibition correlated well with ribosome depurination, consistent with the inability of the depurinated ribosomes to translate protein. In contrast, translation inhibition did not correlate with cytotoxicity, indicating that translation inhibition does not entirely account for the cytotoxicity of RTA. These results were different from those observed with yeast expressing a single chain RIP, pokeweed antiviral protein (PAP). Ribosome depurination did not lead to translation inhibition in yeast expressing several nontoxic PAP mutants, including N70A (36), suggesting possible differences in the way translation is inhibited by PAP and ricin.

Cell growth rate does not always correlate with ribosome depurination. The rate of growth was measured by examining the doubling time of the mutants. As shown in Table 1, the doubling time of cells expressing preRTA was 18 h, while cells harboring the vector control had a doubling time of 6.3 h. The doubling times of cells expressing the active site mutant, E177K, or the nontoxic mutants, G140R and ΔI184, were longer than cells harboring the empty vector, even though these mutants did not depurinate ribosomes or inhibit translation. Although ribosomes were depurinated and translation was inhibited in cells expressing the double mutant, P95L-E145K, the doubling time of cells expressing this mutant (10 h) was similar to the doubling time of cells expressing the active site mutant E177K (9.8 h). In contrast, the doubling time of cells expressing S215F (15 h), which depurinated ribosomes and inhibited translation, was similar to cells expressing the wild type preRTA, although this mutant was nontoxic. These results demonstrated that the rate of growth of yeast cells containing the RTA mutants did not always correlate with the extent of ribosome depurination, indicating that the reduction in growth is not entirely due to ribosome depurination.

Characteristic markers of apoptosis are observed in cells expressing the preRTA. The previous results indicated that the reduction in growth observed in cells expressing preRTA was not entirely due to ribosome depurination or translation inhibition. To assess whether cell death induced by expression of preRTA, was accompanied by morphological features of apoptosis, apoptotic markers in yeast expressing several RTA mutants were examined. Cells expressing the wild type preRTA, S215F and P95L-E145K were analyzed, since these mutants depurinated ribosomes at a similar level. Cells expressing the active site mutant, E177K, and the double mutant, P250L-A253V, were used as negative controls, since these mutants were not toxic and did not depurinate ribosomes. The single mutants corresponding to each double mutant were used as positive controls, since they were toxic and depurinated ribosomes.

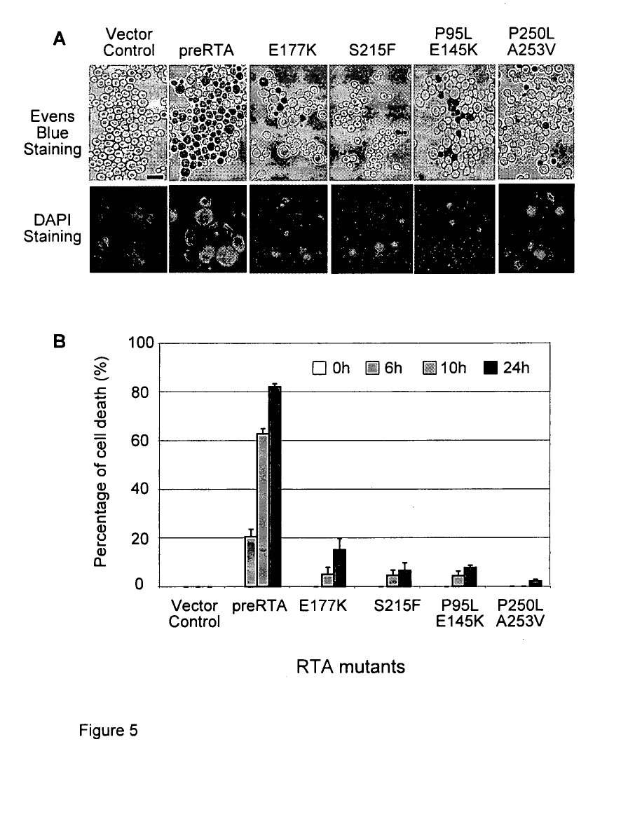

Cells growing in liquid culture were stained with Evans blue at different times after induction. The extent of staining at 24 h post induction is shown in FIG. 5A and is quantified in FIG. 5B. In cells expressing the wild type preRTA or the toxic mutants, cell death was observed at 6 h after induction and gradually increased up to 24 h (FIGS. 5A, 5B). In contrast, minimal loss of cell viability was observed in cells expressing the nontoxic mutants or in cells harboring the empty vector up to 24 h after induction (FIGS. 5A, 5B). These results correlated well with the plate viability assays (FIG. 2).

Chromatin condensation and DNA fragmentation are typical markers for apoptosis in yeast (26). DAPI staining of the cells expressing the nontoxic mutants or harboring the vector showed a normal and single round shaped nucleus, whereas cells expressing the wild type preRTA or the toxic mutants revealed abnormally shaped and fragmented nuclear phenotype at 24 h post induction (FIG. 5A).

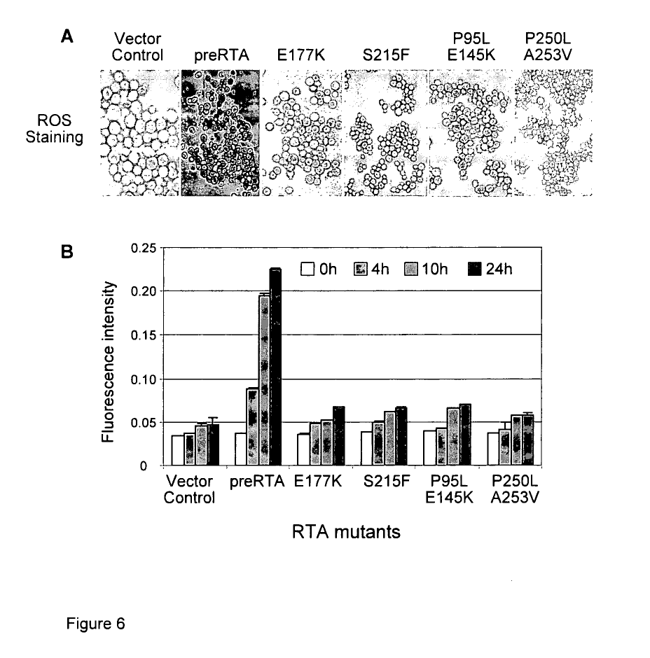

The accumulation of reactive oxygen species (ROS) is a significant trigger of apoptosis in yeast (25). To determine whether yeast cell death induced by ricin is accompanied by the production of ROS, cells were stained with diaminobenzidine (DAB) and visualized under a Zeiss Axiovert 200 inverted microscope at 24 h post induction (FIG. 6). In cells expressing the nontoxic mutants or harboring the vector, there was no staining for ROS up to 24 h post induction (FIG. 6A). In contrast, DAB staining became visible at 6 h after induction in cells expressing preRTA or the toxic mutants, E145K, P250L and A253V and increased up to 24 h (data not shown). These results suggested that expression of the wild type preRTA resulted in increased ROS accumulation and promoted apoptosis-like cell death in yeast.

To quantify intracellular ROS production, 2′, 7′-Dichlorodihydrofluorescein diacetate (DCDHF-DA) oxidation was used as a marker to measure the intracellular level of H₂O₂. As shown in FIG. 6B, the increased level of H₂O₂ observed in cells expressing the preRTA up to 24 h post induction correlated well with cell death and ROS staining (FIG. 6A). In contrast, the H₂O₂ levels did not increase in cells expressing the nontoxic RTA mutants up to 24 h after induction.

These results indicated that RTA expression induced oxidative damage in yeast cells, leading to increased ROS levels. Taken together the results indicated that apoptotic-like cell death was induced in yeast expressing preRTA and correlated well with increased generation of ROS. In contrast, apoptotic-like cell death and production of ROS were not observed in yeast expressing the nontoxic forms of RTA.

Data from additional experiments conducted along the foregoing lines are set forth in TABLE 3.

SITE-DIRECTED MUTATIONS

| Mutation | Parental template | Number | Toxicity | Depurination |
|---|---|---|---|---|
| I249* | pre-RTA | 924 | N | No |
| P250* | pre-RTA | 925 | N | No |
| I251* | pre-RTA | 926 | N | No |
| I252* | pre-RTA | 927 | N | No |
| A253* | pre-RTA | 896 | N | Yes |
| L254* | pre-RTA | 897 | N | No |
| M255* | pre-RTA | 898 | N | No |
| V256* | pre-RTA | 910 | N | No |
| Y257* | pre-RTA | 911 | T | Yes |
| L248A | pre-RTA | 1177 | T | |
| I249A | pre-RTA | 1163 | T | |
| I251S | pre-RTA | 923 | T | Yes |
| I251A | pre-RTA | 1129 | N | No |
| I252A | pre-RTA | 1134 | T | Yes |
| I252S | pre-RTA | 1130 | T | Yes |
| A253R | pre-RTA | 1132 | T | Yes |
| A253V | pre-RTA | 1133 | T | Yes |
| A253N | pre-RTA | 1128 | T | |
| L254A | pre-RTA | 1131 | T | Yes |
| M255A | pre-RTA | 1143 | T | |
| M255L | pre-RTA | 1164 | T | |
| M255R | pre-RTA | 1092 | T | Yes |
| V256R | pre-RTA | 956 | T | Yes |
| V256N | pre-RTA | 1142 | T | |
| M255L V256N | pre-RTA | 957 | N | No |
| M255* | mature RTA | 1175 | N | |
| V256* | mature RTA | 1178 | N | |
| Y257* | mature RTA | 1176 | T | |
| M255L V256N | mature RTA | 1179 | N | |

### REFERENCES

1. Allen, S. C., A. Byron, J. M. Lord, J. Davey, L. M. Roberts, and G. Ladds. 2005. Utilization of the budding yeast Saccharomyces cerevisiae for the generation and isolation of non-lethal ricin A chain variants. Yeast 22:1287-97.
2. Atlas, R. M. 2002. Bioterrorism: from threat reality. Annu Rev Microbiol 56:167-85.
3. Audi, J., M. Belson, M. Patel, J. Schier, and J. Osterloh. 2005. Ricin poisoning: a comprehensive review. JAMA 294:2342-51.
4. Balzan, R., K. Sapienza, D. R. Galea, N. Vassallo, H. Frey, and W. H. Bannister. 2004. Aspirin commits yeast cells to apoptosis depending on carbon source. Microbiology 150:109-15.
5. Bolognesi, A., and L. Polito. 2004. Immunotoxins and other conjugates: pre-clinical studies. Mini Rev Med Chem 4:563-83.
6. Castelletti, D., G. Fracasso, S. Righetti, G. Tridente, R. Schnell, A. Engert, and M. Colombatti. 2004. A dominant linear B-cell epitope of ricin A-chain is the target of a neutralizing antibody response in Hodgkin's lymphoma patients treated with an anti-CD25 immunotoxin. Clin Exp Immunol 136:365-72.
7. Endo, Y., and K. Tsurugi. 1987. RNA N-glycosidase activity of ricin A-chain. Mechanism of action of the toxic lectin ricin on eukaryotic ribosomes. J Biol Chem 262:8128-30.
8. Endo, Y., and K. Tsurugi. 1988. The RNA N-glycosidase activity of ricin A-chain. The characteristics of the enzymatic activity of ricin A-chain with ribosomes and with rRNA. J Biol Chem 263:8735-9.
9. Fahrenkrog, B., U. Sauder, and U. Aebi. 2004. The S. cerevisiae HtrA-like protein Nma111p is a nuclear serine protease that mediates yeast apoptosis. J Cell Sci 117:115-26.
10. Frankel, A., D. Schlossman, P. Welsh, A. Hertler, D. Withers, and S. Johnston. 1989. Selection and characterization of ricin toxin A-chain mutations in Saccharomyces cerevisiae. Mol Cell Biol 9:415-20.
11. Frankel, A., P. Welsh, J. Richardson, and J. D. Robertus. 1990. Role of arginine 180 and glutamic acid 177 of ricin toxin A chain in enzymatic inactivation of ribosomes. Mol Cell Biol 10:6257-63.
12. Griffiths, G. D., C. D. Lindsay, A. C. Allenby, S. C. Bailey, J. W. Scawin, P. Rice, and D. G. Upshall. 1995. Protection against inhalation toxicity of ricin and abrin by immunization. Hum Exp Toxicol 14:155-64.
13. Hartley, M. R., and J. M. Lord. 2004. Cytotoxic ribosome-inactivating lectins from plants. Biochim Biophys Acta 1701:1-14.
14. Hudak, K. A., A. B. Hammell, J. Yasenchak, N. E. Turner, and J. D. Dinman. 2001. A C-terminal deletion mutant of pokeweed antiviral protein inhibits programmed+1 ribosomal frameshifting and Ty1 retrotransposition without depurinating the sarcin/ricin loop of rRNA. Virology 279:292-301.
15. Hudak, K. A., B. A. Parikh, R. Di, M. Baricevic, M. Santana, M. Seskar, and N. E. Turner. 2004. Generation of pokeweed antiviral protein mutations in Saccharomyces cerevisiae: evidence that ribosome depurination is not sufficient for cytotoxicity. Nucleic Acids Res 32:4244-56.
16. Hur, Y., D. J. Hwang, O. Zoubenko, C. Coetzer, F. M. Uckun, and N. E. Turner. 1995. Isolation and characterization of pokeweed antiviral protein mutations in Saccharomyces cerevisiae: identification of residues important for toxicity. Proc Natl Acad Sci USA 92:8448-52.
17. Iordanov, M. S., D. Pribnow, J. L. Magun, T. H. Dinh, J. A. Pearson, S. L. Chen, and B. E. Magun. 1997. Ribotoxic stress response: activation of the stress-activated protein kinase JNK1 by inhibitors of the peptidyl transferase reaction and by sequence-specific RNA damage to the alpha-sarcin/ricin loop in the 28S rRNA. Mol Cell Biol 17:3373-81.
18. Kitaoka, Y. 1998. Involvement of the amino acids outside the active-site cleft in the catalysis of ricin A chain. Eur J Biochem 257:255-62.
19. Knight, B. 1979. Ricin—a potent homicidal poison. Br Med J 1:350-1.
20. Komatsu, N., T. Oda, and T. Muramatsu. 1998. Involvement of both caspase-like proteases and serine proteases in apoptotic cell death induced by ricin, modeccin, diphtheria toxin, and pseudomonas toxin. J Biochem (Tokyo) 124:1038-44.
21. Laun, P., A. Pichova, F. Madeo, J. Fuchs, A. Ellinger, S. Kohlwein, I. Dawes, K. U. Frohlich, and M. Breitenbach. 2001. Aged mother cells of Saccharomyces cerevisiae show markers of oxidative stress and apoptosis. Mol Microbiol 39:1166-73.
22. Ligr, M., F. Madeo, E. Frohlich, W. Hilt, K. U. Frohlich, and D. H. Wolf. 1998. Mammalian Bax triggers apoptotic changes in yeast. FEBS Lett 438:61-5.
23. Lord, J. M., E. Deeks, C. J. Marsden, K. Moore, C. Pateman, D. C. Smith, R. A. Spooner, P. Watson, and L. M. Roberts. 2003. Retrograde transport of toxins across the endoplasmic reticulum membrane. Biochem Soc Trans 31:1260-2.
24. Madeo, F., E. Frohlich, and K. U. Frohlich. 1997. A yeast mutant showing diagnostic markers of early and late apoptosis. J Cell Biol 139:729-34.
25. Madeo, F., E. Frohlich, M. Ligr, M. Grey, S. J. Sigrist, D. H. Wolf, and K. U. Frohlich. 1999. Oxygen stress: a regulator of apoptosis in yeast. J Cell Biol 145:757-67.
26. Madeo, F., E. Herker, S. Wissing, H. Jungwirth, T. Eisenberg, and K. U. Frohlich. 2004. Apoptosis in yeast. Curr Opin Microbiol 7:655-60.
27. Montanaro, L., S. Sperti, A. Mattioli, G. Testoni, and F. Stirpe. 1975. Inhibition by ricin of protein synthesis in vitro. Inhibition of the binding of elongation factor 2 and of adenosine diphosphate-ribosylated elongation factor 2 to ribosomes. Biochem J 146:127-31.
28. Monzingo, A. F., and J. D. Robertus. 1992. X-ray analysis of substrate analogs in the ricin A-chain active site. J Mol Biol 227:1136-45.
29. Morris, K. N., and I. G. Wool. 1994. Analysis of the contribution of an amphiphilic alpha-helix to the structure and to the function of ricin A chain. Proc Natl Acad Sci USA 91:7530-3.
30. Morris, K. N., and I. G. Wool. 1992. Determination by systematic deletion of the amino acids essential for catalysis by ricin A chain. Proc Natl Acad Sci USA 89:4869-73.
31. Munishkin, A., and I. G. Wool. 1995. Systematic deletion analysis of ricin A-chain function. Single amino acid deletions. J Biol Chem 270:30581-7.
32. Narayanan, S., K. Surendranath, N. Bora, A. Surolia, and A. A. Karande. 2005. Ribosome inactivating proteins and apoptosis. FEBS Lett 579:1324-31.
33. O'Hare, M., L. M. Roberts, P. E. Thorpe, G. J. Watson, B. Prior, and J. M. Lord. 1987. Expression of ricin A chain in Escherichia coli. FEBS Lett 216:73-8.
34. Olsnes, S., and J. V. Kozlov. 2001. Ricin. Toxicon 39:1723-8.
35. Osborn, R. W., and M. R. Hartley. 1990. Dual effects of the ricin A chain on protein synthesis in rabbit reticulocyte lysate. Inhibition of initiation and translocation. Eur J Biochem 193:401-7.
36. Parikh, B. A., U. Baykal, R. Di, and N. E. Turner. 2005. Evidence for retro-translocation of pokeweed antiviral protein from endoplasmic reticulum into cytosol and separation of its activity on ribosomes from its activity on capped RNA. Biochemistry 44:2478-90.
37. Parikh, B. A., C. Coetzer, and N. E. Turner. 2002. Pokeweed antiviral protein regulates the stability of its own mRNA by a mechanism that requires depurination but can be separated from depurination of the alpha-sarcin/ricin loop of rRNA. J Biol Chem 277:41428-37.
38. Piatak, M., J. A. Lane, W. Laird, M. J. Bjorn, A. Wang, and M. Williams. 1988. Expression of soluble and fully functional ricin A chain in Escherichia coli is temperature-sensitive. J Biol Chem 263:4837-43.
39. Rao, P. V., R. Jayaraj, A. S. Bhaskar, O. Kumar, R. Bhattacharya, P. Saxena, P. K. Dash, and R. Vijayaraghavan. 2005. Mechanism of ricin-induced apoptosis in human cervical cancer cells. Biochem Pharmacol 69:855-65.
40. Simpson, J. C., L. M. Roberts, K. Romisch, J. Davey, D. H. Wolf, and J. M. Lord. 1999. Ricin A chain utilizes the endoplasmic reticulum-associated protein degradation pathway to enter the cytosol of yeast. FEBS Lett 459:80-4.
41. Stirpe, F., and M. G. Battelli. 2006. Ribosome-inactivating proteins: progress and problems. Cell Mol Life Sci. 63:1850-1866.
42. Thordal-Christensen, H., Z. Zhang, Y. Wei, and D. B. Collinge. 1997. Subcellular localization of H₂O₂ in plants. H₂O₂ accumulation in papillae and hypersensitive response during the barley-powdery mildew interaction. The Plant J. 11:1187-1194.
43. Tommasi, M., D. Castelletti, M. Pasti, G. Fracasso, I. Lorenzetti, S. Sartoris, C. Pera, G. B. Ferrara, G. Tridente, and M. Colombatti. 2001. Identification of ricin A-chain HLA class II-restricted epitopes by human T-cell clones. Clin Exp Immunol 125:391-400.
44. Turner, N. E., D. J. Hwang, and M. Bonness. 1997. C-terminal deletion mutant of pokeweed antiviral protein inhibits viral infection but does not depurinate host ribosomes. Proc Natl Acad Sci USA 94:3866-71.
45. Turner, N. E., B. A. Parikh, P. Li, and J. D. Dinman. 1998. The pokeweed antiviral protein specifically inhibits Ty1-directed+1 ribosomal frameshifting and retrotransposition in Saccharomyces cerevisiae. J Virol 72:1036-42.
46. Vitetta, E. S., J. E. Smallshaw, E. Coleman, H. Jafri, C. Foster, R. Munford, and J. Schindler. 2006. A pilot clinical trial of a recombinant ricin vaccine in normal humans. Proc Natl Acad Sci USA 103:2268-73.
47. Xu, Q., J. M. Jurgensmeier, and J. C. Reed. 1999. Methods of assaying Bcl-2 and Bax family proteins in yeast. Methods 17:292-304.

### INDUSTRIAL APPLICABILITY

The present invention has utility at least in the fields of cancer therapy and bioterrorism defense.

All patent and non-patent publications cited in this specification are indicative of the level of skill of those skilled in the art to which this invention pertains. All these publications and patent applications are herein incorporated by reference to the same extent as if each individual publication or patent application was specifically and individually indicated to be incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A ricin mutant that is altered relative to a ricin A chain toxin having an amino acid sequence of SEQ ID NO:1 (RTA 1-267), wherein the mutant lacks the C-terminal 12-20 amino acid residues of RTA 1267.

2. The ricin mutant of claim 1, which is (RTA 1-248).

3. The ricin mutant of claim 1, which is (RTA 1-249).

4. The ricin mutant of claim 1, which is (RTA 1-250).

5. The ricin mutant of claim 1, which is (RTA 1-250, P250L).

6. The ricin mutant of claim 1, which is (RTA 1-251).

7. The ricin mutant of claim 1, which is (RTA 1-253).

8. The ricin mutant of claim 1, which is (RTA 1-254).

9. The ricin mutant of claim 1, which is (RTA 1-255).

10. A ricin mutant that is altered relative to a ricin A chain toxin having an amino acid sequence of SEQ ID NO:1 (RTA 1-267), wherein the mutant is noncytotoxic to eucaryotic cells and exhibits eucaryotic ribosome depurination activity at least about equal to that of the ricin A chain toxin having the amino acid sequence of RTA 1-267.

11. The ricin mutant of claim 10, wherein said mutant contains the mutation S215F or the double mutation P95L, E145K, or wherein said mutant lacks RTA 253-267).

12. The ricin mutant of claim 10, which is RTA 1-267, S215F.

13. The ricin mutant of claim 10, which is RTA 1-267, P95L, E145K).

14. The ricin mutant of claim 10, which is RTA 1-252.

15. A ricin mutant that is altered relative to a ricin A chain toxin having an amino acid sequence of SEQ ID NO:1 (RTA 1-267), wherein the mutant is noncytotoxic to eucaryotic cells, and contains the mutation G83D, G140R, A147P, E208K, 1251A, P202L or R213D, or the double mutation P250L, A253V or M255L, V256N.

16. The ricin mutant of claim 15, which is (RTA 1-267, G83D), (RTA 1-267, G140R), (RTA 1-267, A147P), (RTA 1-267, E208K), (RTA 1-267, 1251A), (RTA 1-202, P202L), (RTA 1-267, P250L, A253V), (RTA 1-267, M255L, V256N), or (RTA 1-213, R213D).

17. A composition comprising area ricin mutant having an amino acid sequence of SEQ ID NO:1 (RTA 1-267), wherein the mutant lacks the C-terminal 12-20 amino acid residues of RTA 1-267, and a carrier.

18. A nucleic acid molecule having a sequence encoding a ricin mutant having an amino acid sequence of SEQ ID NO:1 (RTA 1-267), wherein the mutant lacks the C-terminal 12-20 amino acid residues of RTA 1-267.

19. A composition comprising the nucleic acid of claim 18, and a carrier.

20. The composition of claim 17, which is a vaccine, and the ricin mutant is present in an amount effective to elicit an immune response to ricin A chain toxin in an animal.

21. The composition of claim 19, which is a vaccine, and wherein the nucleic acid expresses the ricin mutant in an amount effective to elicit an immune response to ricin A chain toxin in an animal.

22. The composition of claim 17, wherein the ricin mutant is present in an amount effective to elicit a therapeutic response in a cancer patient.

23. The composition of claim 17, wherein the ricin mutant is conjugated to a ligand that specifically binds a receptor on a cancer cell.

24. A method of eliciting an immune response to ricin A chain toxin, comprising contacting an animal with a ricin mutant having an amino acid sequence of SEQ ID NO:1 (RTA 1-267), wherein the mutant lacks the C-terminal 12-20 amino acid residues of TRA 1-267, in an amount effective to elicit an immune response to ricin A chain toxin, wherein an immune response to ricin A chain toxin is elicited in the animal.

25. A method of eliciting an immune response to ricin A chain toxin, comprising contacting an animal with the nucleic acid of claim 18, in an amount effective to express the ricin mutant in an amount effective to elicit an immune response to ricin A chain toxin, wherein an immune response to ricin A chain toxin is elicited in the animal.

26. A method of treating cancer, comprising administering to a cancer patient a therapeutically effective amount of a ricin mutant having an amino acid sequence of SEQ ID NO:1 (RTA 1-267), wherein the mutant lacks the C-terminal 12-20 amino acid residues of RTA 1-267.
